(19) United States
(12) Patent Application Publication
(10) Pub. No.: US 2019/0040262 A1
(43) Pub. Date: Feb. 7, 2019

(54) METHOD FOR PROVIDING AN AMOUNT OF PIGMENT

(71) Applicant: GLANZSTOFF INDUSTRIES GMBH, St. Pölten (AT)

(72) Inventors: Bernhard MÜLLER, Baden (AT); Stepanka JANKOVA, Litom??ice (CZ); Enrique HERRERO ACERO, Wien (AT); Georg M. GÜBITZ, Hart bei Graz (AT)

(21) Appl. No.: 16/076,576

(22) PCT Filed: Feb. 16, 2017
(86) PCT No.: PCT/EP2017/000223

§ 371 Date: Aug. 8, 2018

(30) Foreign Application Priority Data
Feb. 18, 2016 (DE) 10 2016 001 910.7

(51) Int. Cl. C09B 67/02; C09B 67/42; C12P 19/14; C12P 7/10; C12P 19/12; C12P 19/02; D01F 2/14; D01F 1/04; D01F 1/07

(52) U.S. Cl. CPC C09B 67/0097; C09B 67/0071; C12P 19/14; C12P 7/10; D01F 1/07; C12P 19/02; D01F 2/14; D01F 1/04; C12P 19/12

(57) ABSTRACT

The invention relates to a method for providing an amount of pigment in which, when glycosidic bonds of a polysaccharide structure are cleaved, pigments contained in the polysaccharide structure, in particular pigments comprising color, luminous and/or flame-retardant pigments, are released and the amount of pigment is formed by the pigments released in this manner.

### CROSS REFERENCE TO RELATED APPLICATION

The present application is a 35 U.S.C. § 371 national phase entry application of, and claims priority to, International Patent Application No. PCT/EP2017/000223, filed Feb. 16, 2017, which claims priority to German Patent Application No. DE 102016001910.7, filed Feb. 18, 2016, the disclosures of which are hereby incorporated by reference in their entirety for all purposes.

### BACKGROUND

The invention relates to a method for providing an amount of pigment and also a cellulosic fibre, particularly regenerated fibre, having a pigment obtained from it.

It is known that there are numerous pigments, with which products from the most varied fields of application may be given additional properties. These include colour pigments for example, which may give a product a desired colour, but also flame-retardant pigments for example, which may ensure flame resistance of the products.

An example of the latter is the Exolit product range of the well-known manufacturer Clariant, which may be applied in numerous fields in its variations, in the plastics industry or also the textile industry for example, in which a particular advantage is also seen in the freedom from halogen of this product. In this the production of Exolit for example is carried out on an industrial scale in industrial plant and the amounts of pigment produced in this way may be supplied to the user in the desired amounts.

### DETAILED DESCRIPTION

The invention is based on the task of simplifying a method of the type indicated at the beginning, particularly considering the cost/benefit overall.

This task is solved by the invention through a further development of the method of the type indicated at the beginning, which essentially is characterised in that pigments contained/held in a polysaccharide structure are released by cleaving (splitting) the glycosidic bonds of the polysaccharide structure and the amount of pigment is formed from the pigments released in this way.

In this the invention is based on the knowledge that with polysaccharide structures that contain pigments a source of pigments is available, which may be tapped into in spite of the non-water solubility or very bad water solubility of the pigments as a rule and also if applicable their high chemical stability with regard to physical effects such as changes in pH value or higher temperatures in the way of release free from destruction, so that an amount of pigment may be formed and provided from the pigments released.

In a preferred variation of the method the polysaccharide structure is split into several substructures when cleaving. This speeds up the method for extracting pigment.

Preferably it is also provided that disaccharide is split from an end of the chain of the structure and/or substructure(s) when cleaving. Therefore the yield of the pigment that may be achieved is increased, extracting the pigment is also speeded up.

In a further preferred embodiment glucose is formed when cleaving. The number of different end products occurring when extracting pigment may be reduced further in this way, which makes utilisation of the end products easier.

In a particularly preferred embodiment cleaving is carried out particularly at normal pressure at a temperature of less than 100° C., preferably less than 80° C., particularly less than 60° C. This simplifies extracting the pigment with regard to plant technology, as no measures have to be taken to handle high working temperatures.

In a further preferred embodiment work is carried out at room temperature type temperatures, therefore preferably at temperatures below 34° C., preferably below 31° C., particularly below 28° C. This allows methods that are energy efficient.

In a particularly preferred design of the method cleaving is carried out enzymatically by hydrolysis, particularly by means of hydrolases of EC category 3.2.1.x. The enzymes used may reduce the activation energy necessary for cleaving the glycosidic bonds.

In this preferably an embodiment is imagined, in which the polysaccharide structure is cellulosic and has and/or consists of cellulose II in particular. In this the crystallinity of the polysaccharide structure may be over 20%, particularly preferably over 28% but below 60%, particularly preferably below 40%. In this connection it is provided that endoglucanase (3.2.1.4) and/or exoglucanase (3.2.1.91), particularly also p-glucosidase (3.2.1.21), is/are used for cleaving. Through the first a larger number of ends of the chain of the structure may be produced, on which the second mentioned may work simultaneously, whilst the third mentioned that may be used may hydrolyse cellubiose into glucose. In addition further peptide sequences, which have no catalytic activity themselves, such as hydrophobins for example, may be added to the peptides indicated above to speed up the reaction further.

Preferably the hydrolases used contain those of the GH61 family. In a particularly preferred embodiment an enzyme mixture is used, the activity of which has at least 80%, preferably at least 90%, particularly at least 95% of the activity of Cellic CTec3, measured in BHU(2)/g product, Cellic CTec3 may be used in particular.

Preferably the polysaccharide structure/regenerated cellulose structure is contained in a body, particularly of textile origin, which may be crushed, particularly mechanically, into part bodies below a preset size before cleaving. Apart from the polysaccharide/regenerated cellulose structure, the body may contain further structures, particularly wool or fibres made of synthetic materials, such as polyester, polyamide 6, polyamide 6.6, aromatic polyamides (m- and p-aramide), polybenzimidazole (PBI), p-phenyl-2,6-benzobisoxazole (PBO), polyimide, polyimide amide, modacrylic, melamine, elastane or combinations of them for example.

Therefore it has been recognised that, particularly in textile bodies, often cellulose structures are contained, which contain pigments. Certainly the latter are present only in a rather small proportion in relation to a-cellulose, between about 1 to 20% by weight for example, but precisely when flame-retardant pigments are used, the proportion in relation to the cellulose may also be 20 to 30% by weight and even more, if there is a combination of pigments acting differently for example. Therefore “amount of pigment” in the sense of the claim is to be understood not only as an amount of a specific pigment, but also these pigment mixtures made of different pigments, an organophosphorous pigment and a colour and/or luminous pigment for example. There may also be dulling pigments, pigments absorbing or reflecting infrared radiation or X-rays or also waxy phase change materials and ground up ion exchangers contained as pigments. Examples of using pigments of this type are found in AT 513426, AT 508687 A1, AT 511638 A1, AT 509801 A1 or AT 510229 B1 for example.

With regard to the size of the part bodies present after mechanical crushing, dimensions of less than 150 mm, preferably less than 70 mm, particularly less than 15 mm are used, with fibres as part bodies these dimensions would be preferred fibre lengths.

As already indicated above, the inclusion of pigments contained in the cellulose II or regenerated cellulose structure is caused, particularly by being spun in, when producing cellulosic fibres, in which the body containing the structure has fibres of this kind or shapes produced from them.

In a useful design of the method cleaving is carried out in an aqueous solvent, in which the body/part bodies containing the structure are dispersed. The precise composition of the solvent is of minor importance, but it should be a solvent that is suitable for the enzyme used and may be based on water in particular. Preferably work is carried out at a pH of 6 or lower, preferably 5.4 or lower, particularly 5.0 or lower and particularly 4 or higher, preferably 4.4 or higher.

After dispersing the mechanically crushed bodies or cut material, the cellulases used may be added while stirring at the process temperatures set.

Particularly preferably the dispersion is stirred/shaken, particularly at 60 rpm or higher, preferably at least 80 rpm or higher, for more than 24 hours, preferably more than 32 hours, particularly more than 40 hours or even 48 hours.

However, mechanical crushing of the bodies or structures containing the pigments is not absolutely necessary. A medium containing cellulase for example may be brought into contact with these bodies/structures continuously, passed around them or passed by them for example. The bodies/structures may be treated according to a method of the immersion bath type in particular.

Furthermore, separation of the solvent with the saccharide residue, which is still present only in the form of glucose in particular, is carried out usefully for forming the amount of pigment.

Furthermore, preferably it is provided that the saccharide residue contained in the solvent, present in the form of glucose in particular, is subject to further conversion, preferably conversion into ethanol by means of fermentation. With reference to this it is useful to remove further substances contained in the solvent beforehand, such as cellulases for example and byproducts if applicable, which were also contained in the polysaccharide structure due to the process technology used, such as zinc ions for example. The separation methods known to the person skilled in the art are available for this.

The solid of the dispersion containing the pigments may be isolated, washed with water for example and run through a drying process in particular. Isolation of the solid of the dispersion containing the pigments may also be carried out by a process of washing with organic solvents for example.

Furthermore, in order to reduce the reaction time with the cellulase, the bodies containing the polysaccharide structure/regenerated cellulose may be subject to further swelling/activation in acid or alkaline conditions before hydrolysis.

Furthermore, the invention protects a method for producing a cellulosic fibre, particularly regenerated fibre, in which pigments, particularly from one or several types of flame-retardant pigments, luminous pigments, colour pigments and dulling pigments, are contained in the cellulose matrix of fibres, particularly by being spun in, during production, particularly in the viscose method or modified viscose method, characterised in that the pigments used for this are provided at least partly by a method according to one of the aspects of extracting pigment indicated above. However, other pigments may also be used, including those also indicated above. Also fibres other than viscose fibres may also be imagined, such as lyocell fibres for example. Furthermore, it is imagined that other types of fibres are also used again, such as mo-dal, carbamate or cuprammonium fibres or fibres spun from ionic liquids for example. Apart from this, the use of substances that form fibres other than cellulose, such as α-1,3-glucan for example, is also imagined for forming the polysaccharide structure. This is conceivable alone or also in combination with cellulose.

Furthermore, a (man-made) fibre containing cellulose II is protected, particularly cellulosic regenerated fibre, with pigments contained in the cellulose matrix of the fibres, which essentially is characterised in that the pigments stem at least partly from an amount of pigment provided according to one of the aspects of extracting pigment explained above.

In this preferably at least 5%, also preferably at least 15%, of the total pigments used stem from the amounts of pigment provided according to the invention.

An example procedure for providing pigment according to the invention is explained in the following:

In a first step mechanical crushing of a cellulosic initial body is carried out. This may be carried out by a shredder, a cutting wheel, a cutting mill or a hammer mill for example, in which fibres are cut to a fibre length of 10 mm for example.

Optionally, if wished, activation of the cut material in acid or alkaline conditions may take place at this point.

The cut material is dispersed in a solvent that is suitable for the cellulases used, particularly water.

The cellulases, which preferably contain at least endoglucanase and exoglucanase, are added while stirring at a process temperature of 50° C. for example while stirring, in which stirring may be carried out for 24 hours at 100 rpm for example.

Then separation of the solvent that is rich in glucose is carried out and isolation of the pigment or pigment mixture, as a rule through one or several filtration steps (also through centrifuging) and extraction, as are well known as such to the person skilled in the art. A sedimentation method may also be used when separating.

If centrifuging is used when separating, preferably the gravitational field of the centrifuge is more than 1000 g, preferably more than 2000 g, particularly more than 3000 g.

If present in the initial body used, the solvent may also contain natural or synthetic fibres, which are hardly or not attacked or affected structurally by enzymatic hydrolysis. These may also be separated from the solvent, in which a particular separation step may be used. These fibres may also be supplied for reuse in this way.

After washing the pigment or pigment mixture and drying it, the amount of pigment is provided and may be used for the desired application. A flame-retardant pigment provided with the method according to the invention may be used again in the viscose spinning method and spun into regenerated cellulose fibres for example.

Any byproducts from the fibres for example, compounds that contain amine or Zn ions for example, may be removed before further use by means of ion exchangers or reduced to a desired size. Alternatively or in addition to the ion exchangers, ultrafiltration may also be used to remove them. Removing zinc ions and also the enzymes in the reaction solution by means of ultrafiltration for example is particularly preferred, if the solvent that is rich in glucose, as explained above, is subject to fermentative treatment for example. Because inhibition of the growth of microorganisms used for fermentation is avoided in this way, which otherwise may occur due to the toxicity of zinc ions for example.

The following concrete examples of embodiments of the method according to the invention also make clear the advantages achieved with the invention compared with hydrolysis catalysed by acid that destroys pigments.

### Example 1

1.0 g of Viscont® FR fibres (10 mm long), which contain 18% by weight of Exolit 5060, are added to 49.5 mL of an aqueous buffer solution (citric acid 50 mM pH=4.8). Then 0.5 mL of Cellic CTec3 (Novozymes) were added and shaken at 50° C. and 100 rpm for 24 hours. Already after 10 hours almost complete release of the fibres has occurred and the pigments released are present in colloidal form. The pigment was separated from the reaction solution by means of gravity filtration until the filtrate was clear. The colourless residue remaining in the filter was dried to a weight constancy (169 mg, 94% of the theoretical yield).

### Example 2

Like for example 1, however, the reaction mixture is shaken at 22° C. (room temperature) and 100 rpm for 72 hours. Already after 48 hours extensive release of the fibres has occurred and the pigments released are present in colloidal form. There is complete hydrolysis after 72 hours. Processing was carried out like for example 1 (89% theoretical yield).

### Example 3

1.0 g of Viscont® FR day light yellow fibres (10 mm long), as described under EP 2 714 972 B1, which contain 19% by weight of Exolit 5060 and 3% of a day light pigment (Yellow 10G), are added to 49.5 mL of an aqueous buffer solution (citric acid 50 mM pH=4.8). Then 0.5 mL of Petrozyme STR6 (Petrohofer) were added and shaken at 50° C. and 100 rpm for 24 hours. Already after 10 hours almost complete release of the fibres has occurred and the pigments released are present in colloidal form. Processing was carried out like for example 1.

### Example 4

Like for example 3, however, the reaction mixture is shaken at 22° C. (room temperature) and 100 rpm for 72 hours. Already after 48 hours extensive release of the fibres has occurred and the pigments released are present in colloidal form. There is complete hydrolysis after 72 hours. Processing was carried out like for example 1.

### Comparative Example

5 g of Viscont® FR fibres (10 mm), which contain 18% by weight of Exolit 5060, are put in an Erlenmyer flask and then aqueous sulphuric acid (of differing concentration) added and stirred at room temperature (23° C.) for the duration of the test.

TABLE 1
Hydrolysis tests with sulphuric acid

| Type of fibre | Viscont ® FR | | Viscont ® FR | |
|---|---|---|---|---|
| Water | 400 | mL | 100 | mL |
| Acid | 5 | g | 5 | g |
| Concentration | 5M | | Concentrated | |
| Duration | 48 | hours | 1 | hour |

| | | |
|---|---|---|
| Yield | Fibre not released | Fibre becomes black in colour |

The invention is not limited to the above examples. Rather the characteristics of the following claims and those of the above explanations may be essential for putting the invention into effect in its various embodiments.

1. A method for providing an amount of pigment in which, when glycosidic bonds of a polysaccharide structure are cleaved, pigments contained in the polysaccharide structure, in particular pigments comprising colour, luminous and/or flame-retardant pigments, are released and the amount of pigment is formed by the pigments released in this manner.

2. The method according to claim 1, in which the polysaccharide structure is split into several substructures when cleaving.

3. The method according to claim 1, in which disaccharide is split from an end of the chain of the structure and/or substructure when cleaving.

4. The method according to claim 1, in which glucose is formed when cleaving.

5. The method according to claim 1, in which cleaving is carried out at a temperature of less than 100° C., preferably less than 80° C., particularly less than 60° C.

6. The method according to claim 1, in which cleaving is carried out enzymatically by hydrolysis.

7. The method according to claim 1, in which the polysaccharide structure has and/or consists of cellulose II, particularly regenerated cellulose.

8. The method according to claim 6, in which endoglucanase and/or exoglucanase, particularly also p-glucosidase, is used for cleaving.

9. The method according to claim 7, in which the polysaccharide structure/cellulose 11 structure/regenerated cellulose structure is contained in a body, particularly of textile origin, which is crushed, particularly mechanically, into part bodies below a preset size before cleaving.

10. The method according to claim 7, in which the polysaccharide structure/cellulose 11 structure/regenerated cellulose structure is contained in a body, particularly of textile origin, which contains further structures, particularly fibres made of wool or synthetic materials, such as polyester, polyamide 6, polyamide 6.6, aromatic polyamide, polybenzimidazole, p-phenyl-2,6-benzobisoxazole, polyimide, polyimide amide, modacrylic, melamine, elastane or combinations of them for example.

11. The method according to claim 9, in which the inclusion of pigments contained in the cellulose 11 structure is caused, particularly by being spun in, when producing them by cellulosic fibres, which form a/the body containing the cellulose 11 structure.

12. The method according to claim 1, in which cleaving is carried out in an aqueous solvent, in which the body/part bodies containing the structure are dispersed.

13. The method according to claim 12, in which separation of the solvent with the saccharide residue, particularly glucose, is carried out after cleaving.

14. The method according to claim 13, in which the separated saccharide residue, particularly the glucose, is converted into ethanol by means of fermentation.

15. The method according to claim 1, in which the formation of the amount of pigment contains a process for washing the pigments isolated.

16. The method according to claim 9, in which activation of the mechanically crushed body containing the polysaccharide structure is carried out reducing the reaction time with the cellulase.

17. A method for producing a cellulosic fibre, particularly regenerated fibre, in which pigments, particularly from one or several types of flame-retardant pigments, luminous pigments and colour pigments, are contained in the cellulose matrix of the regenerated fibre, particularly by being spun in, during production, particularly in the viscose method or modified viscose method, characterised in that the pigments used for this are provided at least partly by the method according to claim 1.

18. The method of claim 17, wherein the cellulosic fibre, particularly regenerated fibre, with pigments contained in the cellulose matrix of the fibre, is characterised in that the pigments stem at least partly from an amount of pigment provided by the method according to claim 1.

19. The method of claim 1, wherein the pigment is provided by the method according to claim 1 for providing items with a property imparted by the pigment, particularly in the textile industry, particularly for producing cellulosic fibres in particular, preferably regenerated cellulose fibres, and flat shapes produced from such fibres for textile use.
